(19) United States
(12) Patent Application Publication
(10) Pub. No.: US 2021/0138302 A1
(43) Pub. Date: May 13, 2021

(54) ACTIVITY TRACKING AND MOUNTING APPARATUS AND METHOD FOR USE WITH ACTIVITY TRACKING VIRTUAL WALK E-COMMERCE MOBILE APPLICATION

(71) Applicant: ROSS PURDIE, TOKYO (JP)

(72) Inventor: ROSS PURDIE, TOKYO (JP)

(21) Appl. No.: 16/861,078

(22) Filed: Apr. 28, 2020

Related U.S. Application Data
(60) Provisional application No. 62/933,837, filed on Nov. 11, 2019.
(60) Provisional application No. 62/972,991, filed on Feb. 11, 2020.
(60) Provisional application No. 63/001,006, filed on Mar. 27, 2020.

(51) Int. Cl. A63B 24/00; A61B 5/11; A61B 5/00; A61B 5/024; A63B 23/035; A63B 21/00; A63B 23/04

(52) U.S. Cl. CPC A63B 24/0062; A61B 5/1118; A61B 5/4866; A61B 5/0002; A61B 5/6887; A61B 5/024; A63B 2071/0644; A63B 21/151; A63B 21/4034; A63B 23/04; A61B 2503/12; A63B 2220/833; A63B 2220/62; A63B 23/03525

(57) ABSTRACT

The present invention pertains to an activity tracking apparatus, desk mounting system and mobile application combined with associated software with sensory hardware and telemetry equipment that can monitor activity, via an activity administration module of an assemblage of sensors and electronic monitors. The electronic module or modules may be interfaces with application software resident on a PC or portable devices. The mounting system is made up of a series of a desk that has evenly spaced integrity holes drilled to the bottom of a tabletop, whereby brackets containing several hooks can be manually assembled to allow for customizable ergonomic configuration for the user. The mobile application can incorporate incentives through the use of virtual reality and e-commerce components that can increase user engagement and motivation to meet fitness goals. The present invention may also interface with wearable technology such as watches to monitor personal health conditions during periods of activity.

### PRIORITY CLAIMS

This application claims the benefit of U.S. Provisional Patent Application Ser. No. 62/933,837 filed on Nov. 11, 2019; U.S. Provisional Patent Application Ser. No. 62/972,991 filed on Feb. 11, 2020; and U.S. Provisional Patent Application Ser. No. 63/001,006 filed on Mar. 27, 2020, all of which are incorporated herein by reference.

### BACKGROUND OF THE INVENTION

It is well known that consistent and frequent physical activity is essential to maintaining health. However, due to developments in many work environments, the reality is that the level of physical activity required for optimal health is not met due to the common practice of sitting down for long periods of time, i.e., when working at a desk. It is now known that sitting for lengthy periods of time is the cause of many physical ailments and an increased early mortality rate. Even if an individual consistently exercises, sitting for long periods of time can still result in a higher risk of muscular skeletal disorders, obesity, diabetes, cancer, and heart disease.

Sitting for extended periods also puts the average person at risk for bad posture, poor blood circulation and weight gain. If one remains in the same position for a prolonged period of time, the human body misses crucial opportunities for physical activity, leading to poor blood circulation and a lack of metabolic activity. All of these reasons make it important for anyone with a desk job to make time for physical activity every hour or so. A standing desk is one way to avoid the negative consequences of sitting for too long. To reduce the risk of these health ailments, office workers can take frequent exercise breaks, do some sitting exercises at their desks, or use a balance ball to encourage proper posture. However, these methods can be distracting and cause discomfort, resulting in significantly reduced employee productivity.

The economic impact of the declining health of employees that sit for long periods of time is also a major concern of many corporations. The cost of providing essential employee benefits such as health insurance significantly increases and starts to negatively impact profit margins. It becomes evident that there is a need to improve employee health through non-distracting preventative methods.

Current desk solutions that provide increased activity levels can also be damaging to furniture. Desks that are re-configured to allow for a more active sitting experience are also not easily customized to fit the needs of multiple users. Furniture is often damaged as it has to be re-configured post manufacturing, and the cost to do so is also a notable expense. For example, the cost of a standing desk can be two or three times the price of a standard office desk.

There is a great need to provide incentive and motivation in order to engage a user for continued use of a fitness system. Current fitness systems and corresponding applications that track fitness activity may provide incentive by keeping track of activity data and user progress. It has been shown that greater incentive and user engagement can be generated by allowing the user to multi-task through options that extend beyond those of fitness and physical health, such as adding an e-commerce or entertainment component in tandem with the process of increasing one's activity level.

The economic impact of the declining health of employees that sit for long periods of time is also a major concern of many corporations. The cost of providing essential employee benefits such as health insurance significantly increases and starts to negatively impact profit margins. It becomes evident that there is a need to improve employee health through non-distracting preventative methods.

### SUMMARY OF THE INVENTION

The present invention pertains to an activity tracking apparatus and associated software with sensory hardware and associated telemetry equipment which actively monitors sitting activity, via a sitting activity administration module of assemblage of sensors and electronic monitors. The electronic module or modules may be interfaces with application software resident on a PC or portable device such as a tablet, smartphone or cloud. The present invention functions by acting as a foot swing that can be used while sitting in a chair. The present invention is suspended from either the bottom of a desk, or stand, forcing the user to balance when their feet is mounted to the apparatus. This results in slight conscious and unconscious movements that encourage blood circulation and muscle activity.

In one embodiment, the present invention incorporates a desk mounting system for an activity tracking apparatus that is able to actively monitor sitting activity, via an assemblage of sensors and electronic monitors. In one embodiment, the activity tracking apparatus is suspended in such as way so as to not destroy the integrity of the desk. The activity tracking apparatus is mounted flat against the bottom of the desk in order to avoid noticeable and potentially dangerous protrusion of the apparatus. This is considered a low-profile mounting system that can easily be reconfigured without the use of tools such as screwdrivers or handheld drills.

In another embodiment, a series of integrity holes are pre-drilled into a wood composite or particle board desk. The holes are evenly distributed on the underside of the desk so as to allow for multiple configurations of mounting the brackets that will hold the activity tracker in place and allow it to be customized to an ideal ergonomic position for each user.

The present invention uses easily moveable mounting brackets that consists of several hooks from which to suspend the activity tracking apparatus in a customized ergonomic position for the user. The bracket is mounted into pre-drilled integrity holes located on the bottom of a desk using a manually adjustable screw that is placed through the holes of the bracket to secure it to the pre-drilled integrity holes of a desk. The manually adjustable screw does not require additional tools to be installed. These mounting brackets can be secured freely in more than 150 configurations under the desk. This mounting system proves to be durable over time as it maintains the integrity of the desk.

In one embodiment, the activity tracking apparatus can be affixed to the bottom of the desk using 2 brackets and a tightened steel wire. The apparatus is mounted in such a way so as to be predictable to the foot movement of the user. The steel wire provides flexibility similar to a cantilever. This mounting system also allows for the height of the mounted apparatus to be adjustable in order to provide ergonomic comfort.

In one embodiment, the mountable activity tracking apparatus features adjustable height options to be customized to each user. The activity tracking apparatus allows for each user to have a separate profile in a corresponding mobile application. The application is paired with a sensor that confirms the user of the activity tracking apparatus at a specific desk. The user can tap on the sensor ID to verify their data log, as every desk sensor has a unique ID.

The electronic module or modules may be interfaces with application software resident on a PC or portable device such as a tablet, smartphone or cloud. The present invention functions by acting as a foot swing that can be used while sitting in a chair. The present invention is suspended from either the bottom of a desk, or stand, forcing the user to balance when their feet is mounted to the apparatus. This results in slight conscious and unconscious movements that encourage blood circulation and muscle activity.

The present invention monitors user movement throughout the day. The present invention can also analyze the activity data to calculate the ratio of the work metabolic rate to the resting metabolic rate (MET) and daily calorie expenditure. This activity tracking data can be viewed by the user as a gauge to improve one's health on a daily basis, through activity data analytics.

The present invention incorporates activity tracking sensors that are mounted to the device and used for data collection and as a Bluetooth or Wi-Fi enabled controller. This data is collected through an online platform and displayed on an online or mobile application interface. This data can also be synchronized with wearable fitness tracking devices and stored on mobile health tracking applications such as those commonly found on a smartphone device. The activity data tracked by the present invention can also be synchronized to social media platforms and applications, allowing the user to post and share activity data. This can result in increased motivation and frequency of physical activity and achieving individual health goals.

The movements performed on the apparatus equate to as much as a 20% increase in caloric burn, and a 20 to 30% increase in one's metabolic rate. The MET calculations of using the present invention are 1.4×. The benefits of implementing the present invention in the workplace include: increased overall health and energy levels of employees; improved attention span; improved focus; and increased productivity of employees. Increased activity through the use of the present invention also results in increased employee mindfulness and health awareness, which contributes to overall work satisfaction and endorphin production while sitting. These benefits ultimately result in reduced costs of healthcare and an increase in company profits.

One embodiment of the present invention is the software functionality to detect, count and track user steps. The source code of the software of the present invention can recognize the full completions of the 4 basic apparatus movements of steps: forwards and backwards; left and right; circular or helicopter movement; and marching movement. The present invention incorporates both iOS and Android mobile applications that store daily movement data, steps, METs and caloric burn of the individual. This data is also stored on a cloud server for administrative purposes. Program options like virtual walks are available to the user through the mobile application, and can be customized to individual preferences. All step, METs and calorie calculation data will be stored on a daily basis, and reset to zero at 24:00 PM local time.

In another embodiment, a virtual reality based mobile application is used to encourage movement in conjunction with using the activity tracking apparatus. This application combines movement tracking with e-commerce as a motivational tool that provides enhanced user engagement. This mobile application can be implemented by employers and business owners, allowing for access to every employee or individual working for the employer's company and using the activity tracking apparatus. This mobile application shall be integrated with a corporate project management tool and will capture the productivity of each individual or employee. Hence, using activity tracking apparatus will not only help employers in monitoring their employee's health, but will also benefit them in determining the employee's productivity. This activity tracking mobile application shall be configured for employers in such a way that by its unique algorithm it can be customized to track the productivity of each employee based on parameters that are set by the employer.

In one embodiment, the mobile application program can be used in tandem with a physical activity tracking apparatus. The mobile application can track the physical steps accumulated by a user using an activity tracking apparatus that is being used during normally stationary activities, such as sitting at a desk. The activity tracking apparatus that is able to actively monitor sitting activity, via an assemblage of sensors and electronic monitors. This data is collected electronically and linked to a mobile application that keeps track of the user's activity. In the preferred embodiment of the present invention, the mobile application that keeps track of user activity data can also incorporate additional incentives through the use of virtual reality and e-commerce components.

The activity tracking mobile application can be used as a motivational tool for employees by providing user engagement, reducing work related stress, and preventing many diseases caused by long office hours and sitting. The engagement experience is created through a virtual walk through of various locations, such as of various vacation destinations or the user's favorite location situated anywhere in the world. The user is also able to shop online while on the virtual street walking experience. Additional incentives include offering the user discounts on various items using the application's unique reward programs. These rewards can be obtained and accumulated by using activity tracking apparatus and meeting specific goals, as well as referring others to use the activity tracking apparatus and mobile application.

Through the use of the activity tracking mobile application, users will get suggestions of nearby destinations, such as: hotels; restaurants; shops; and art galleries. Based upon these suggestions, the user can go to these locations virtually by implementing virtual reality street views. Subsequently, the user can shop online by virtually browsing the items from stores accessed during that virtual walk. Moreover, the user will also be able to do direct video calling to the shop's sales executives and be able to see the items in real time.

E-commerce store owners can have access to the mobile application's activity tracker portal at their end, where they will be able to offer virtual browsing to the user. This virtual browsing shall be facilitated by technology such as software beacons, and other technology platforms that would be associated with the e-commerce tool in the mobile and web applications. The user can experience shopping in real-time by being able to view the product virtually. The user would shop using the virtual street walk platform, and earn special discounts from the e-commerce stores as a reward for using the activity tracking apparatus and it's software platform. By using the activity tracking apparatus with the application for a maximum number of hours, and by earning rewards through new user referrals, the user can achieve greater health benefits, shop virtually and secure great discounts as well.

In another embodiment, activity detection and tracking capabilities of the present invention can be used for tactile or upper body movement on a desk such as activity detection and tracking implemented into a computer mouse. Utilizing the gyro and accelerometer, in a Bluetooth and Wi-Fi enabled environment, to move the cursor by manipulating the apparatus of the present invention, to replace the mouse. Input, or cursor stabilization will be done as one click on the PC's space key. Double clicking will act as a toggle to highlight the cell, or grab a section of text or photo. These cursor functions can also be integrated for use with software commonly used in the workplace such as Microsoft Word, Excel or Outlook. This results in the user becoming more engaged with work tasks, and the use of the mouse results in less distraction. This implementation is ergonomically beneficial as it will eliminate the common risk of developing Carpal tunnel syndrome, a condition that occurs when the median nerve is squeezed or compressed as it travels through the wrist and causes pain, numbness, and tingling in the hand and arm.

In another embodiment, the present invention can be implemented in other tactile or hand co-ordination implements such as game controllers. This will allow for the utilization of the free range of motion which can be produced from the random movement derived from the present invention to make it possible for combined sensor activity tracking to be an improvement on existing game controllers. Utilizing both Gyro and Accelerometer functions of the present invention, gameplay can be improved in terms of entertainment for the user as well as increased physical activity, resulting in an increase in the user's blood circulation while the user is manipulating the controller for their game.

These and other aspects, objects, features and advantages of the present invention, are specifically set forth in, or will become apparent from, the following detailed description of an exemplary embodiment of the invention.

### BRIEF DESCRIPTION OF THE DRAWINGS

FIG. 1 is a rendering of the present invention and its sensors.

FIG. 2 is a diagram of the different movements or steps that are detected and tracked by the present invention.

FIG. 3 is a drawing of the on-board sensors of the present invention.

FIG. 4 depicts an administration page so that group managers can monitor the activity of a group of people.

FIG. 5 is an image of the desk mounting system of the present invention.

FIG. 6 is an image of a mounting bracket of the present invention.

FIG. 7 is a screen flow diagram of the account activation process of the present invention.

FIG. 8 is a screen flow diagram of the user details process of the present invention.

FIG. 9 is an image of the application demo animation screen of the present invention.

FIG. 10 is an image of the account navigation screen of the present invention.

FIG. 11 is a screen flow diagram of the application tools navigation menu of the present invention.

FIG. 12 is an image of the user workout preference screen of the present invention.

FIG. 13 is a screen flow diagram of the user BMR calculator module of the present invention.

FIG. 14 is a screen flow diagram of the user macro calculator module of the present invention.

FIG. 15 is a screen flow diagram of the user diet tool module of the present invention.

FIG. 16 is a screen flow diagram of the e-commerce navigation module of the present invention.

FIG. 17 is a screenshot of the virtual walk module of the present invention.

FIG. 18 is a screenshot of the hotel selection option of the present invention.

FIGS. 19A-F are screenshots of the virtual walk module of the present invention.

### DETAILED DESCRIPTION OF THE PREFERRED EMBODIMENT

FIG. 1 is a rendering of the present invention and its sensors. In accordance with the preferred embodiment of the present invention, the activity tracking apparatus 100 can be connected to a stand 102 or mounted under a desk or table. The activity tracking apparatus 100 is equipped with up to four battery powered Bluetooth enabled sensors 104. These sensors 104 are equipped with a gyroscope and accelerometer to detect movement, direction and speed.

FIG. 2 is a diagram of the different movements or steps that are detected and tracked by the present invention. In accordance with the preferred embodiment of the present invention, the activity tracking apparatus can detect, count and track user steps 200. The present invention can recognize the full completions of the four basic apparatus movements of steps 200: forwards and backwards 202; left and right 204; circular or helicopter movement 206; and marching movement 208.

FIG. 3 is a drawing of the on-board sensors of the present invention. In accordance with the preferred embodiment, the present invention is equipped with up to 4 battery powered Bluetooth enabled sensors 300. The circuit board 302 of each sensor 300 contains: a push button 304; low energy Bluetooth processing unit 306; an RGB LED light 308; and a 9-axis inertial measurement unit (IMU) barometer sensor 310. Users can also customize the circuit board 302 by adding additional optional sensing capabilities 312. Other components of the sensor 300 include: an ambient light sensor 314; a temperature sensor 316; an ARM Cortex M0 microprocessor 318; a coin cell battery 320; and on-board flash-memory. Other on-board sensors can include a triple-axis gyroscope, accelerometer, and magnetometer, and humidity sensor.

FIG. 4 depicts an administration page so that group managers can monitor the activity of a group of people. In accordance with the preferred embodiment of the present invention, the active sitting administration module 400 aggregates, analyzes and displays activity data for multiple users. The module consists of a home screen 402, data grouped by various users or teams 404, and module settings 406. The administrator 410 can view alerts and messages 408 based on parameters that can be customized in the module settings 406. The module administrator 410 can group users by teams 404, and view the activity data for each of these teams 404. Each user displayed in a specific team page is listed as a member 412. The administrator 410 can view data for each team member 412 that includes: usage 414 data based on the total time that the activity apparatus was used; the date 416 of usage; the amount of steps 418 as detected by the sensors; calories 420 expended through the use of the activity apparatus; and the calculated distance 422 of user steps. Each team page 400 also shows the total aggregated activity 424 of the team for each month based on the average number of steps.

The Active sitting administration module can be used by employers to monitor the activity of employees. Use of the activity tracking apparatus can be incentivized by a points system based on the activity data of each user. Points can be earned by the activity data aggregated from the activity tracking apparatus as well as other activity data collected from a user's smartphone device or wearable tracking technology, such as the amount of daily steps that are detected by smartphone pedometer tracking applications and wearable pedometers. User biometric data, such as electrocardiogram (EKG) data that monitors heartbeat activity, can also be collected and turned into additional points. These points act as an activity incentive and can be redeemed for certain goods and services, such as free items or discounts. The aggregated activity data and incentive points program of the present invention can also be synchronized with user social media accounts. Users can share activity data and points, as well as participate in group chats and messaging, on various social media platforms as an additional incentive to boost user activity levels.

FIG. 5 is an image of the desk mounting system of the present invention. In accordance with the preferred embodiment of the present invention, the underside of a desk or tabletop 500 has integrity holes 502, which are drilled into the wood composite or particleboard material. The holes 502 are evenly distributed on the underside of the desk 500 so as to allow for multiple configurations of mounting the brackets 504 that will hold the activity tracker in place and allow it to be customized to an ideal ergonomic position for each user. The outlined area 506 of the desk 500 shows the approximate location and size of the activity tracking apparatus in relation to the placement of the mounting brackets 504 in the integrity holes 502.

FIG. 6 is an image of a mounting bracket of the present invention. In accordance with the preferred embodiment of the present invention, the mounting bracket 600 consists of several hooks 602 from which to suspend the activity tracking apparatus in a customized ergonomic position for each user. The bracket is mounted into pre-drilled integrity holes located on the bottom of a desk using a manually adjustable screw 606 that is placed through the holes of the bracket 604 to secure it to the pre-drilled integrity holes of a desk. The manually adjustable screw 606 does not require additional tools to be installed. These mounting brackets 600 can be secured freely in more than 150 configurations under the desk without the need for special tools.

FIG. 7 is a screen flow diagram of the account activation process of the present invention. In accordance with the preferred embodiment of the present invention, the user account creation and activation process of the activity tracking application begins with a splash screen that features a video animation in the background and some text introducing the user to the product. The user is then offered an option to select the desired language first and also, an option to either Login (for existing users) and Sign Up (in case the user is new to the application). If users click at Login, they are taken to the login screen to enter their existing login credentials or their social media credentials. This page will also have a sign-up option for new user accounts. If the user registers a new account, they are taken to a sign-up screen that lets the new user to insert his/her details and create a new login for the present invention. Owing to the ever-enhancing technology in the market, the users will have an option to activate their face ID for quick and easy login as well. Lastly, the user is also able to access a screen that lets them reset their password. At this screen, the user needs to insert his/her registered email and will receive a link at the registered email to reset his/her password.

FIG. 8 is a screen flow diagram of the user details process of the present invention. In accordance with the preferred embodiment of the present invention, once the users sign-in successfully, they will be taken to the screen to insert their personal details such as name, age, date of birth, weight, height and gender. Once they click on the Create Account option, they will be taken to the next screen where they will be required to insert their phone number. After entering the mobile number, the user will receive a one-time passcode (OTP) on his/her mobile and will be taken to the next screen for entering the OTP and verifying user contact details.

FIG. 9 is an image of the application demo animation screen of the present invention. In accordance with the preferred embodiment of the present invention, once the user has completed the account set up process, the user will then view the application demo animation, which gives the user a general introductory overview of the mobile application and all of its functions.

FIG. 10 is an image of the account navigation screen of the present invention. In accordance with the preferred embodiment of the present invention, once the user has finished viewing the application demo animation, the user will see a series of tabs that allows the user to access account information such as: my records; friends; refer and earn; account settings; leaderboard; e-commerce; social; tools; and issues. Each tab may have a multiple number of screens attached to it.

Once the user clicks on the tools tab, they will have access to the various tools available at their disposal. The examples of available tools are: progress tracker; diet tools; training tools; water reminder; BMR calculator; macro calculator; and body fat calculator. The sample screens, as to how the user will be able to navigate through the available tools, are displayed in FIGS. 11, 12 and 13.

FIG. 11 is a screen flow diagram of the application tools navigation menu of the present invention. In accordance with the preferred embodiment of the present invention, the Progress Tracker tool gives access to the user progress based on the vitals inserted by the user and the steps run through the activity tracking apparatus.

FIG. 12 is an image of the user workout preference screen of the present invention. In accordance with the preferred embodiment of the present invention, the user may insert his/her workout preference in the workout preference tool screen. This screen will offer options like walking very little, walking 1-3 hours per day, 3-5 hours per day or more than 5 hours per day based on user's fitness requirements to burn a certain amount of calories. Based on these preferences and taking precedence from the BMR calculation, the training tool will have a snapshot of recommended combination of the activity tracking apparatus movements based on user vitals and the required calories to be burnt.

FIG. 13 is a screen flow diagram of the user BMR calculator module of the present invention. In accordance with the preferred embodiment of the present invention, the BMR Calculator tool lets the user calculate their body to mass ratio and total energy expenditure (TEE).

FIG. 14 is a screen flow diagram of the user macro calculator module of the present invention. In accordance with the preferred embodiment of the present invention, based on the TEE calculated in the BMR tab, the macro calculator will offer various diet plan combinations to the user. The user will be able to select low/medium/high carbs/protein/fat rich diet per their dietary preferences.

FIG. 15 is a screen flow diagram of the user diet tool module of the present invention. In accordance with the preferred embodiment of the present invention, once the users have selected the diet combination per their macro calculation, they will be able to choose a diet plan for themselves. The Diet Tool will enable the users to create their own diet plan based on the calorie intake required by the user per day and also, based on the diet combination selected in the macro tab.

FIG. 16 is a screen flow diagram of the e-commerce navigation module of the present invention. In accordance with the preferred embodiment of the present invention, the user will be able to have the following e-commerce options through the proposed application. Here, the user will have options displayed such as: mobile recharge; DTH/internet recharge/payment; utility bill payment; tax payment; and insurance premium payment. Moreover, the user will have options to redeem their accumulated rewards points at stores featured on the application, which would be accumulated to their activity tracking application account either by referring a friend or from achieving their set targets as a result of the virtual walk feature.

FIG. 17 is a screenshot of the virtual walk module of the present invention. In accordance with the preferred embodiment of the present invention, this screenshot shows the virtual reality walk as though the user is walking through the streets of a specific location, while linked to the activity tracking apparatus as a physical means to generate the steps necessary to walk through the streets displayed on the mobile application screen. This allows for the user to also get suggestions of the nearby attractions such as hotels, restaurants, art galleries etc. Based upon the suggestions, the user can go to these locations virtually by using street views, as shows in FIG. 17. Subsequently, with providing an e-commerce solution based on activity completed using the activity tracking apparatus. The user can shop for anything online by virtually browsing the items from the shops that are available to the user on that specific route. Moreover, the user can also be able to do direct video calling to the shop's sales executives and be able to see real time item and its description.

In terms of the e-commerce connection to the mobile application, shop owners will have access to the activity tracker portal at their end, where they will be able to offer virtual browsing to the user of the mobile application. This virtual browsing shall be facilitated by the software beacons and other technology platforms that would be associated with our e-commerce tool in the mobile and web applications. The user will have real-time shopping experience by being able to view the product/product quality virtually for themselves. The user can shop using the virtual street walk platform will get additional discount from the shops as a reward of using our device and software platform. This is a unique product, where, by using the activity tracking apparatus for maximum number of hours and by earning rewards on referring their friends and family to use the activity tracking apparatus, the end user can take health benefits, shop virtually and secure great discounts as well.

FIG. 18 is a screenshot of the hotel selection option of the present invention. In accordance with the preferred embodiment of the present invention, this screenshot is a representation of the suggestions made by the virtual reality e-commerce component of the application to make suggestions to the user while on a specific route. This particular screenshot shows suggestions of hotels that are near the user's route location.

FIGS. 19A-F are screenshots of the virtual walk module of the present invention. In accordance with the preferred embodiment of the present invention, when the virtual walk application is in use, the user can simply go to virtual street walk to their desired location. For this they will have to simply select the geolocation street view option from the activity tracking apparatus virtual walk application. For the virtual walk destinations, the user will be able to select the pre-loaded destinations and also the dynamic street view locations of their own choice. They can set goal to walk and burn their calories along with other health benefits offered by the activity tracking apparatus, which is shown in the following sections. This application offers a unique feature, such that the end user will be able to use these street views location using SIRI and Google Assistance as well. The end user will also get automatic updates on their progress towards the set goals for their virtual walk and the health parameters along with other set goals, if any.

FIG. 19A is a screenshot that depicts the choice of location made by the user. This can be done by searching for a location by typing in the desired location or using a voice command prompt to specify the location (such as using a SIRI enabled voice command).

FIG. 19B is screenshot that depicts an overall view of the general location as searched by the user. The user can then zoom in further to choose a specific location in that area to start the virtual walk.

FIG. 19C is screenshot that depicts the virtual walk route selected by or suggested to the user based on parameters specified by the user. This shows the distance and estimated time it would take to complete the virtual walk based on the user's historical activity tracking apparatus data (i.e., the speed of which the user can use the activity tracking apparatus). The user can also add or save this route to a list of favorite routes in the mobile application.

FIG. 19D is screenshot that depicts the virtual walk route selected by or suggested to the user based on parameters specified by the user. This shows the distance and estimated time it would take to drive to the selected location from the current location based on current traffic data.

FIG. 19E is screenshot that depicts the next step in selecting the virtual walk route, whereby an icon is presented to the user on the screen to select the street view of that specific route in order to commence the virtual walk. This shows the distance and estimated time it would take to complete the virtual walk based on the user's historical activity tracking apparatus data (i.e., the speed of which the user can use the activity tracking apparatus).

FIG. 19F is screenshot that depicts the zoomed in map overview of certain sections of the virtual walk that was selected by the user. The user can use this zoomed in view of streets to select specific sections of the virtual walk route in order to create a customized virtual reality walk experience while using the activity tracking apparatus. This allows the user to also choose specific streets with certain shops the user would be interested in browsing in order to integrate the mobile application's e-commerce component.

While various embodiments of the disclosed technology have been described above, it should be understood that they have been presented by way of example only, and not of limitation. Likewise, the various diagrams may depict an example architectural or other configuration for the disclosed technology, which is done to aid in understanding the features and functionality that may be included in the disclosed technology. The disclosed technology is not restricted to the illustrated example architectures or configurations, but the desired features may be implemented using a variety of alternative architectures and configurations. Indeed, it will be apparent to one of skill in the art how alternative functional, logical or physical partitioning and configurations may be implemented to implement the desired features of the technology disclosed herein. Also, a multitude of different constituent module names other than those depicted herein may be applied to the various partitions. Additionally, with regard to flow diagrams, operational descriptions and method claims, the order in which the steps are presented herein shall not mandate that various embodiments be implemented to perform the recited functionality in the same order unless the context dictates otherwise.

Although the disclosed technology is described above in terms of various exemplary embodiments and implementations, it should be understood that the various features, aspects and functionality described in one or more of the individual embodiments are not limited in their applicability to the particular embodiment with which they are described, but instead may be applied, alone or in various combinations, to one or more of the other embodiments of the disclosed technology, whether or not such embodiments are described and whether or not such features are presented as being a part of a described embodiment. Thus, the breadth and scope of the technology disclosed herein should not be limited by any of the above-described exemplary embodiments.

Terms and phrases used in this document, and variations thereof, unless otherwise expressly stated, should be construed as open ended as opposed to limiting. As examples of the foregoing: the term “including” should be read as meaning “including, without limitation” or the like; the term “example” is used to provide exemplary instances of the item in discussion, not an exhaustive or limiting list thereof; the terms “a” or “an” should be read as meaning “at least one,” “one or more” or the like; and adjectives such as “conventional,” “traditional,” “normal,” “standard,” “known” and terms of similar meaning should not be construed as limiting the item described to a given time period or to an item available as of a given time, but instead should be read to encompass conventional, traditional, normal, or standard technologies that may be available or known now or at any time in the future. Likewise, where this document refers to technologies that would be apparent or known to one of ordinary skill in the art, such technologies encompass those apparent or known to the skilled artisan now or at any time in the future.

What is claimed is:

1. An apparatus for exercising a person's legs and tracking activity associated with said exercising including a suspended footrest device for suspension from a fixed location, comprising:
(a) a pair of spaced-apart footrests;
(b) an elongated member extending laterally along the length thereof between said spaced-apart footrests, the elongated member having a pair of opposed ends and a medial section, whereby each of said spaced-apart footrests swings pivotally about a central connector disposed along said medial section of said elongated member so that said footrests are pivotally connected to each other along at least two axes to permit simultaneous relative movement therebetween in at least two planes;
(c) at least one flexible and non-rigid suspension member having a first end connected to the medial section of the elongated member, and a second end connected to the fixed location for suspension of the footrest device therefrom; and
(d) an activity tracker affixed to said footrest device to track activity due to leg movement by said person.

2. The suspended footrest device of claim 1 including a duration counter for measuring the duration of said leg movement according to start and stop input provided by said activity tracker.

3. The suspended footrest device of claim 2 including an electronic interface device for providing said person's smartphone with information indicative of accumulated exercise according to the output of said activity tracker.

4. The suspended footrest device of claim 3 including an electronic interface device for providing calorie counting information to said person.

5. A interface according to claim 1 wherein an ecommerce communication link may be conducted in coordination with said tracking activity.

6. A method for exercising a person's legs and tracking activity associated with said exercising including a suspended footrest device for suspension from a fixed location, comprising:
(a) a pair of spaced-apart footrests;
(b) an elongated member extending laterally along the length thereof between said spaced-apart footrests, the elongated member having a pair of opposed ends and a medial section, whereby each of said spaced-apart footrests swings pivotally about a central connector disposed along said medial section of said elongated member so that said footrests are pivotally connected to each other along at least two axes to permit simultaneous relative movement therebetween in at least two planes;
(c) at least one flexible and non-rigid suspension member having a first end connected to the medial section of the elongated member, and a second end connected to the fixed location for suspension of the footrest device therefrom; and
(d) an activity tracker affixed to said footrest device to track activity due to leg movement by said person.

7. A method for providing and tracking leg exercise by engaging said suspended footrest device of claim 6 including a duration counter for measuring the duration of said leg movement according to start and stop input provided by said activity tracker.

8. A method for providing and tracking leg exercise by engaging said suspended footrest device of claim 7 including an electronic interface device for providing said person's smartphone with information indicative of accumulated exercise according to the output of said activity tracker.

9. A method for providing and tracking leg exercise by engaging said suspended footrest device of claim 8 including an electronic interface device for providing calorie counting information to said person.

10. A method according to claim 6 wherein ecommerce may be conducted in coordination with said tracking activity.

11. A method for exercising a person's legs and tracking activity associated with said exercising including a suspended footrest device for suspension from a fixed location, comprising:
(a) a pair of spaced-apart footrests;
(b) an elongated member extending laterally along the length thereof between said spaced-apart footrests, the elongated member having a pair of opposed ends and a medial section, whereby each of said spaced-apart footrests swings pivotally about a central connector disposed along said medial section of said elongated member so that said footrests are pivotally connected to each other along at least two axes to permit simultaneous relative movement therebetween in at least two planes;
(c) at least one flexible and non-rigid suspension member having a first end connected to the medial section of the elongated member, and a second end connected to the fixed location for suspension of the footrest device therefrom;
(d) an activity tracker affixed to said footrest device to track activity due to leg movement by said person;
(e) a computer interface with existing application software to track a user's human condition including heart rate, amount of activity and duration of said activity; and
(f) memory storage for storing the performance of said user on a day by day basis to track chronological performance and track overall health.

12. A method according to claim 11 wherein ecommerce may be conducted in coordination with said tracking activity.
